(19) United States
(12) Patent Application Publication
(10) Pub. No.: US 2020/0113516 A1
(43) Pub. Date: Apr. 16, 2020

(54) METABOLIC RATE MEASUREMENT APPARATUS AND METHOD THEREOF

(71) Applicant: Vanderbilt University, Nashville, TN (US)

(72) Inventors: Christina Marasco, Nashville, TN (US); Amaury Perez, Nashville, TN (US); Jonathan Ehrman, Nashville, TN (US); Orlando Hoilett, West Lafayette, IN (US)

(21) Appl. No.: 16/601,441

(22) Filed: Oct. 14, 2019

Related U.S. Application Data
(60) Provisional application No. 62/745,445, filed on Oct. 14, 2018.

(51) Int. Cl. A61B 5/00; A61B 5/083; A61B 5/024; A61B 5/1455

(52) U.S. Cl. CPC A61B 5/4866; A61B 5/681; A61B 5/02405; A61B 5/02438; A61B 5/14552; A61B 5/0836

(57) ABSTRACT

The exemplified systems, and method thereof, provide a transdermal or transcutaneous, non-invasive sensor system that measures blood carbon-dioxide levels and blood oxygen levels, via optical sensors, to determine an estimate of metabolic rate to provide a user of the system with a direct, continuous metabolic rate measurement. In some embodiments, the exemplified sensor system employs low-cost components such as LEDs and photodiodes to provide such direct, continuous metabolic rate measurement.

### RELATED APPLICATION

This application claims priority to, and the benefit of, U.S. Provisional Patent Application No. 62/745,445, filed Oct. 14, 2018, entitled “Metabolic Rate Measurement Apparatus and Method Thereof,” which is incorporated by reference herein in its entirety.

### BACKGROUND

Basal metabolic rate (BMR) is a measure of the rate of human metabolism while at rest, whereas total metabolic rate (TMR) comprises BMR in addition to energy expenditure from activity, eating, etc. Both are often quantified as an amount of energy, e.g., calorie, Joule, etc., expended per unit of time.

Metabolic rates are typically calculated via direct and indirect calorimetry. Direct calorimeters work by capturing temperature production in an environmental chamber over an extended period of time (i.e. 24 hours).

Indirect calorimeters work by measuring the amount of oxygen inhaled and carbon dioxide exhaled to estimate the rate of substrate (fats, carbohydrates, proteins, etc.) utilization. Such methodologies can provide accurate measurements but are limited to laboratory and clinic use such as in hospitals and athletic training facilities.

Statistical averages of metabolic rate can also be determined solely based on a person's characteristics such as gender, age, height, weight and mapping such parameters to population averages.

There is still a benefit to assessing body metabolic rate without such limitations.

### SUMMARY

The exemplified systems, and method thereof, provide a non-invasive sensor system that measures blood carbon-dioxide levels and blood oxygen levels, via optical sensors, to determine an estimate of metabolic rate to provide a user of the system with a discrete and/or continuous metabolic rate measurement. As used herein, the term “blood carbon-dioxide levels” (also referred to as “blood carbon-dioxide concentration levels”) can refer to specific or total blood carbon dioxide concentration in blood vessels or capillaries, which can include dissolved carbon dioxide concentration, carbonic acid concentration, bicarbonate ion concentration, and/or protein-bound carbon dioxide concentration, among others. As used herein, the term “blood oxygen levels” (also referred to as “blood oxygen concentration levels”) can refer to specific or total blood oxygen concentration in blood vessels or capillaries, which can include protein-bound oxygen concentration and/or dissolved oxygen concentration, among others. Indeed, the use of optical sensors at a peripheral area of the body (e.g., finger, among other locations discussed herein) reduces the complexity of measurements as compared to gas sensors that may have associated tubing and/or month-piece, such as those in portable indirect calorimeters. In some embodiments, the exemplified sensor system employs low-cost components such as LEDs and photodiodes to provide such discrete and/or continuous metabolic rate measurement.

In an aspect, a method is disclosed of determining metabolic rate measurements, the method comprising receiving, by a processor, on an intermittent basis, at each acquisition, a first set of one or more measurement values derived from measurements using a first non-invasive reflectance- or transmission-based transdermal or transcutaneous sensor located on a device placed on or adjacent to a subject, wherein the first set of one or more measurement values is associated with a measure of total blood oxygen content (e.g. protein-bound oxygen concentration, dissolved oxygen concentration), and wherein the first non-invasive reflectance- or transmission-based transdermal or transcutaneous sensor comprises visible and near-infrared photodiodes; determining, by the processor, a blood oxygen concentration value in the blood from one or more of the first set of measurements; receiving, by the processor, on an intermittent basis, at each acquisition, a second set of measurements from the one or more non-invasive reflectance- or transmission-based transdermal or transcutaneous sensors located on the device placed on or adjacent to the subject, wherein the second set of one or more measurement values is associated with a measure of total blood carbon dioxide concentration in the blood (e.g. dissolved carbon dioxide concentration, carbonic acid concentration, bicarbonate ion concentration, protein-bound carbon dioxide concentration), and wherein the second non-invasive reflectance- or transmission-based transdermal or transcutaneous sensors comprises a mid-infrared photodiode; determining, by the processor, a blood carbon dioxide value from one or more of the second set of measurements; determining, by the processor, on an intermittent basis, a metabolic rate value (e.g., from a look-up table or a transfer function) based on the determined blood oxygen concentration value and the determined blood carbon dioxide concentration value; and outputting, by the processor, at a display of the device, said determined metabolic rate value.

In some embodiments, the method includes storing, by the processor, each of the determined metabolic rate values; and transmitting, by the processor, the stored metabolic rate values to a network (e.g., a cloud service) for subsequent display or analysis.

In some embodiments, the second non-invasive reflectance- or transmission-based transdermal or transcutaneous sensors are part of an array of photodiodes.

In some embodiments, the first non-invasive reflectance- or transmission-based transdermal or transcutaneous sensors are part of an array of photodiodes.

In some embodiments, the first set of one or more measurement values associated with the measure of blood oxygen concentration and the second set of one or more measurement values associated with the measure of blood carbon dioxide concentration are acquired simultaneously to one another.

In some embodiments, the visible and near-infrared LEDs are configured to output predominantly at around a 660-nm wavelength and a 940-nm wavelength, respectively.

In some embodiments, the mid-infrared LED is configured to output predominantly at around a 4.2-μm wavelength.

In some embodiments, the outputted metabolic rate values are used to assess and track subject's nutrition, metabolic disorders (e.g., diabetes), activity level, or weight management.

In some embodiments, the method further includes determining, by the processor, on an intermittent basis, at each acquisition, a heart rate value (e.g., in beats/second or Hz) from the received first set of measurements associated with the measure of blood oxygen concentration level or from the received second set of measurements associated with the measure of blood carbon dioxide concentration level; and outputting, by the processor, at the display of the wearable device or portable diagnostic device, said determined heart rate value (e.g., concurrently or separately with the outputted metabolic rate value).

In some embodiments, the method further includes determining, by the processor, on an intermittent basis, a parameter selected from the group consisting of a heart rate variability parameter and an energy expenditure parameter; and outputting, by the processor, at the display of the device, said parameter.

In another aspect, a system is disclosed comprising one or more reflectance- or transmission-based transdermal or transcutaneous sensors, including a first reflectance- or transmission-based transdermal or transcutaneous sensor comprising visible and near-infrared photodiodes having a wavelength associated with blood oxygen concentration (e.g. total blood oxygen concentration, including oxygenated hemoglobin concentration, and dissolved oxygen concentration) and a second reflectance- or transmission-based transdermal or transcutaneous sensor comprising a mid-infrared photodiode associated with blood carbon dioxide concentration (e.g. total carbon-dioxide concentration, including dissolved carbon dioxide concentration, bicarbonate concentration, and carbonate anion concentration); a processor operatively connected to the one or more reflectance- or transmission-based transdermal or transcutaneous sensors and the display; and a memory operatively connected to the processor, the memory having instructions stored thereon, wherein execution of the instructions by the processor, cause the processor to receive a first set of one or more measurement values derived from measurement of the first non-invasive reflectance- or transmission-based transdermal or transcutaneous sensors having been placed on a first surface location of or adjacent to a person; determine a blood oxygen value from the first set of measurements; receive a second set of one or more measurement values from measurement of the second reflectance- or transmission-based transdermal or transcutaneous sensors; determine a blood carbon dioxide value from the second set of measurements; determine a metabolic rate value (e.g., from a look-up table or a transfer function) based on the determined blood oxygen value and the determined blood carbon dioxide value; and output at the display said determined metabolic rate value. In some embodiments, blood carbon dioxide concentration can be tuned to certain carbon dioxide concentration, e.g., bicarbonate concentration and/or carbonate anion concentration, e.g., in the blood or in the tissue. In some embodiments, blood oxygen concentration can be tuned to certain oxygen concentration, e.g., oxygenated hemoglobin concentration, and dissolved oxygen concentration.

In some embodiments, the instructions, when executed by the processor, further cause the processor to store each of the determined metabolic rate values; and transmit the stored metabolic rate values to a network (e.g., a cloud service) for subsequent display or analysis.

In some embodiments, the second reflectance- or transmission-based transdermal or transcutaneous sensors are part of an array of photodiodes.

In some embodiments, the first reflectance- or transmission-based transdermal or transcutaneous sensors are part of an array of photodiodes.

In some embodiments, the first set of one or more measurement values associated with the measure of blood oxygen concentration and the second set of one or more measurement values associated with the measure of blood carbon-dioxide concentration are acquired simultaneously to one another.

In some embodiments, the visible and near-infrared LEDs are configured to output predominantly at around a 660-nm wavelength and a 940-nm wavelength, respectively.

In some embodiments, the mid-infrared LED is configured to output predominantly at around a 4.2-μm wavelength.

In some embodiments, the instructions, when executed by the processor, further cause the processor to determine a heart rate value from the received first set of measurements associated with the measure of blood oxygen level or from the received second set of measurements associated with the measure of blood carbon dioxide level; and output at the display said determined heart rate value (e.g., concurrently or separately with the outputted metabolic rate value).

In some embodiments, the instructions, when executed by the processor, further cause the processor to determine a parameter selected from the group consisting of a heart rate variability parameter and an energy expenditure parameter; and output at the display of the device said parameter.

In another aspect, a non-transitory computer readable medium is disclosed, the computer readable medium having instructions stored thereon, wherein execution of the instructions, when executed by the processor, cause the processor to receive a first set of one or more measurement values derived from measurement of the first non-invasive reflectance- or transmission-based transdermal or transcutaneous sensor having been placed on a first surface location of a person, wherein the first reflectance- or transmission-based transdermal or transcutaneous sensor comprises visible and near-infrared photodiodes having a wavelength associated with blood oxygen concentration (e.g. total blood oxygen concentration, including oxygenated hemoglobin concentration, dissolved oxygen concentration); determine blood oxygen concentration value from the first set of measurements; receive a second set of one or more measurement values from measurement of the second non-invasive reflectance- or transmission-based transdermal or transcutaneous sensor, wherein the second reflectance- or transmission-based transdermal or transcutaneous sensor comprises a mid-infrared photodiode associated with blood carbon dioxide concentration (e.g. total blood carbon dioxide concentration, including dissolved carbon dioxide concentration, bicarbonate concentration, carbonate anion concentration); determine a blood carbon-dioxide value from the second set of measurements; determine a metabolic rate value (e.g., from a look-up table or a transfer function) based on the determined blood oxygen value and the determined blood carbon dioxide value; and output at the display said determined metabolic rate value. In some embodiments, blood carbon dioxide concentration can be tuned to certain carbon dioxide concentration, e.g., bicarbonate concentration and/or carbonate anion concentration, e.g., in the blood or in the tissue. In some embodiments, blood oxygen concentration can be tuned to certain oxygen concentration, e.g., oxygenated hemoglobin concentration, and dissolved oxygen concentration.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a diagram of a system configured to monitor metabolic rate measurements via optical sensors, in accordance with an illustrative embodiment.

FIG. 2 is a diagram showing the operation of the metabolic rate measurement system of FIG. 1, in accordance with an illustrative embodiment.

FIG. 3 is a diagram of a method of operating the system of FIG. 1 to monitor metabolic rate measurements, in accordance with an illustrative embodiment.

FIG. 4 is a diagram illustrating operation of the blood carbon dioxide LED driver circuit of the system of FIG. 1, in accordance with an illustrative embodiment.

FIG. 5 is a diagram illustrating operation of the blood carbon-dioxide pre-amplifier circuit of the system 100 of FIG. 1, in accordance with an illustrative embodiment.

FIG. 6 is a diagram illustrating operation of the blood carbon-dioxide lock-in amplifier circuit of the system 100 of FIG. 1, in accordance with an illustrative embodiment.

FIG. 7 is a diagram illustrating an operation of the blood carbon dioxide LED driver circuit of the system of FIG. 1, in accordance with an illustrative embodiment.

FIG. 8 is a diagram illustrating an operation of the blood oxygen LED driver circuit of the system 100 of FIG. 1, in accordance with an illustrative embodiment.

FIG. 9 is a diagram illustrating operation of the blood oxygen photodiode(s) circuit, in accordance with an illustrative embodiment.

FIG. 10 is a diagram illustrating operation of the blood oxygen sample-and-hold circuit of the system of FIG. 1, in accordance with an illustrative embodiment.

FIG. 11 is a diagram showing experimental results of photodiode responses to pulsed LED of the system of FIG. 1., in accordance to an illustrative embodiment

FIG. 12 is a diagram showing a photodiode sensor response to near- and mid-IR signals in accordance with an illustrative embodiment.

FIGS. 13A, 13B, 13C, 13D, and 13E show example circuit diagram of exemplary system of FIG. 1, in accordance with an illustrative embodiment.

### DETAILED DESCRIPTION

Before the present methods and systems are disclosed and described, it is to be understood that the methods and systems are not limited to specific methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes. Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that may be performed it is understood that each of these additional steps may be performed with any specific embodiment or combination of embodiments of the disclosed methods.

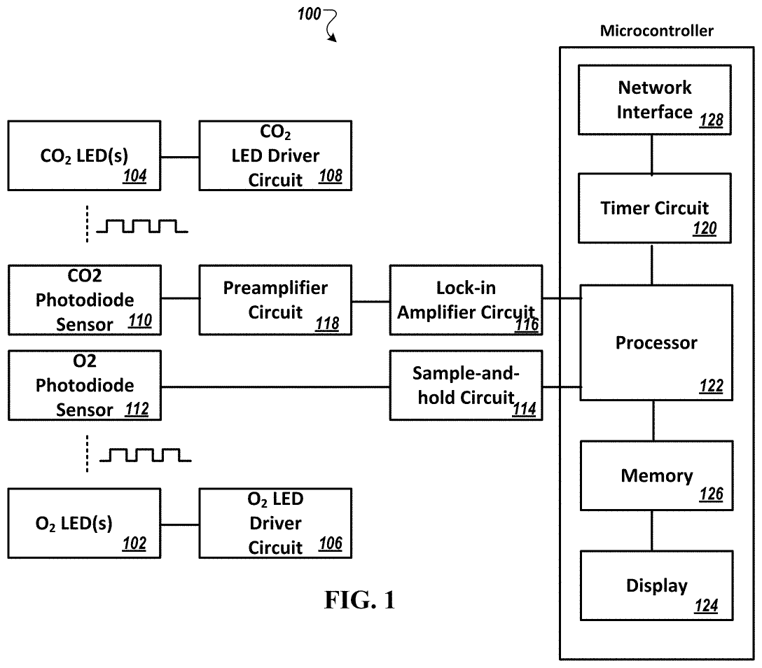

FIG. 1 is a diagram of a system 100 configured to monitor metabolic rate measurements, in accordance with an illustrative embodiment. The system 100 includes, in some embodiments, one or more O₂ LED(s) 102 (also referred to herein as an “blood oxygen LED” 102) and one or more CO₂ LED(s) 104 (also referred to herein as a “blood carbon-dioxide LED” 104) integrated into a wearable device, portable device, or handheld device. In some embodiments, the instant system may be incorporated into pulse oximeter system to display metabolic rate information in conjunction with blood oxygen information. The system 100 includes reflectance- or transmission-based transdermal or transcutaneous sensor s configured as one or more O₂ photodiode sensors 112 (also referred to herein as a “blood oxygen photodiode” 112) and one or more CO₂ photodiode sensors 110 (also referred to herein as an “blood carbon-dioxide photodiode” 110), also integrated into the wearable device, portable device, or handheld device, to receive a reflectance of the output from the corresponding CO₂ LED 104 and O₂ LED 102. The blood oxygen LED(s) 102 is configured to output, in some embodiments, in the visible and near-infrared. The blood carbon dioxide LED(s) 104 is configured to output, in some embodiments, in the mid-infrared.

The system 100, via use of the blood oxygen and blood carbon dioxide LED(s) (e.g., 102, 104) and photodiode sensor(s) (e.g., 112, 110), can detect blood oxygen level (O₂) (e.g., specific or total blood oxygen concentration levels) and blood carbon dioxide level (CO₂) (e.g., specific or total blood carbon dioxide concentration level), respectively, through the skin to provide a more direct, and accurate measure of metabolic rate. From the determined oxygen and carbon-dioxide concentrations, the system 100 can determine a respiratory quotient. The measurement acquired from the sensor and respiratory quotient can be used to calibrate and output an estimated metabolic rate as an indirect calorimetry test.

The blood oxygen photodiode(s) 112 is configured, in some embodiments, to detect the output (or a substantial portion thereof) of the blood oxygen LED(s) 102. In some embodiments, the blood oxygen LED(s) includes two LEDs, one configured to output a 660 wavelength (visible) output and a second configured to output a 940 nm wavelength (near-IR) output. Other wavelengths can be used to detect the O₂ signal. In other embodiments, the blood oxygen LED is configured to output at both the 660 nm wavelength (visible) output and the 940 nm wavelength (near-IR) output based on the waveform that is applied to it.

The output of the blood oxygen photodiode(s) 112 are amplified and digitized to provide as a measure of oxygen levels in the blood stream. In some embodiments, a pulse oximeter and corresponding circuitry is used. The system 100 includes a signal conditioning circuit for the blood oxygen photodiode(s) 112 that includes, in some embodiments, a sample-and-hold circuit 114. In some embodiments, the sample-and-hold circuit 114 comprises a sample-and-hold integrated circuits (ICs) and operational amplifiers (e.g., transimpedance operational amplifier). The sample-and-hold ICs are configured to retain an acquired voltage. The sample-and-hold circuit 114 can serve as a filter to output cleaner signals that are not affected by the rapid change in signals that the photodiodes produce.

The blood carbon dioxide photodiode 110 is configured, in some embodiments, to detect the output of the blood carbon dioxide LED(s) 104 having a detection wavelength, for example, in the mid-IR wavelength. In some embodiments, the detection wavelength is between about 2 μm and about 18 μm. In some embodiments, the detection wavelength is between about 4.1 μm and about 4.5 μm. The output of the blood carbon dioxide photodiode(s) 110 are amplified and digitized to provide as a measure of carbon dioxide levels in the blood stream. The system 100 includes a signal conditioning circuit for the blood carbon dioxide photodiode(s) that includes, in some embodiments, a lock-in amplifier circuit 116 and a preamplifier circuit 118.

In some embodiments, wearable device is configured as a wrist-worn device such as a watch having a wrist strap or as a bracelet, or anklet. In other embodiments, the wearable device is configured as a neck-worn device such as a necklace having a chain. In yet other embodiments, the wearable device is configured with an attachable pin. In yet other embodiments, the wearable device is configured as an arm-worn device, such as an arm-band, or as an ankle-worn device or leg-worn device. In yet other embodiments, the wearable device is configured as an ear- or finger-worn device. In yet other embodiments, the wearable device is configured as a hip-worn or chest strap device. In some embodiments, exemplary sensor system is implemented as a part of a measurement or exercise device, such a scale, an exercise bike, a thread-mill, and the like, for example, in a transdermal or transcutaneous manner to how heart rate is measured at such devices. In some embodiments, the exemplary sensor system is configured to be implemented in a form factor similar to thermometer (e.g., an oral thermometer or a forehead thermometer), a blood pressure cuff, or any other discreate measurement system. In some embodiments, the exemplary sensor system is configured in a video game console.

In some embodiments, the system 100 includes a set of blood oxygen LED(s) 102 and/or a set of blood carbon dioxide LED(s) 104, e.g., each configured in an array. In alternative embodiments, a single set of LEDs is used as the blood oxygen and blood carbon dioxide LEDs (102, 104) to which a single corresponding driver circuit is also used.

The system 100 includes a timer circuit 120 (e.g., a clock circuit) that outputs a timer signal to respective LED drive circuits 106, 108 for the blood oxygen and blood carbon dioxide LEDs (102, 104). In some embodiments, the timer circuit is implemented in a microcontroller.

The processor 122 of the microcontroller is connected to memory 126 having instructions stored thereon, wherein execution of the instructions cause the processor 122 to determine a metabolic rate value from the measured blood oxygen value and the measured blood carbon-dioxide value. The determined metabolic rate value is outputted to a display 124.

In some embodiments, the instructions also cause the processor 122 to determine the heart rate value and variability to co-present with the metabolic rate value. In some embodiments, the instructions also cause the processor 122 to display the measured blood oxygen value and measured blood carbon-dioxide value. In some embodiments, the instructions also cause the processor 122 to determine and output an energy expenditure parameter (e.g., in calories per hour or joules per hour).

In some embodiments, the system 100 can be used as a medical device to assess and track patient nutrition. This may be useful for diagnosing or monitoring metabolic disorders such as diabetes as well as for weight management and for general nutrition analysis. The system 100 is configured, in some embodiments, to measure and output measured heart rate, measured heart rate variability, measured blood oxygen levels, measured blood carbon-dioxide levels, metabolic rate, and energy expenditure. The system can be used to continuously monitor these parameters at the bedside or in a wearable device.

In some embodiments, the system 100 can be used for sports, for example, to monitor diets and health of athletes as a replacement, or a substitute for, inconvenient and expensive measurement system that employs indirect calorimetry, e.g., to track metabolism. With the device's ability to track metabolic rates in real-time, users can also track activities and the time of the day when metabolic rates are at the highest and lowest, allowing athletes to reach their peak performance naturally.

In some embodiments, the system can be used by consumers for weight management and fitness applications, e.g., to monitor diet and fitness routines.

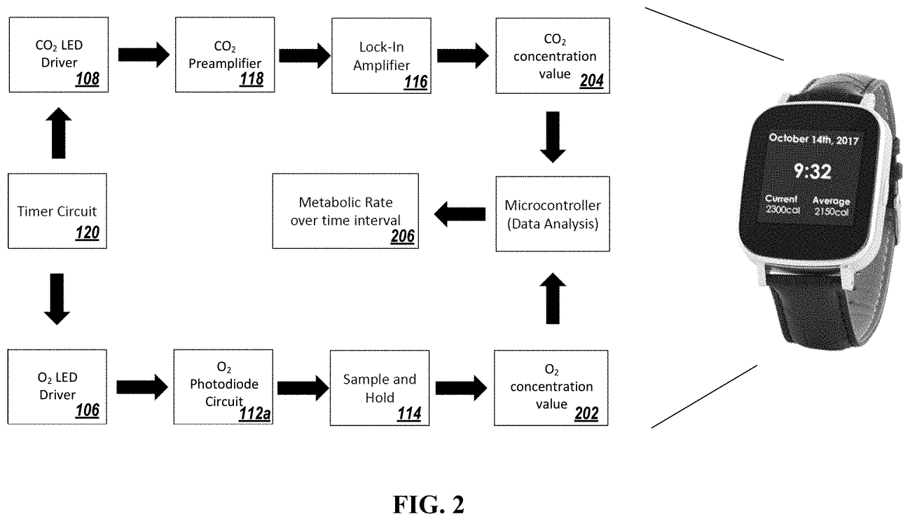

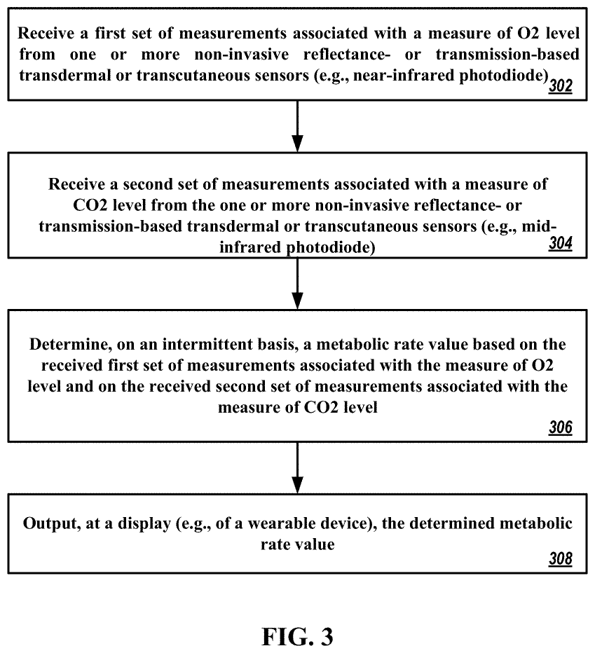

FIG. 2 is a diagram showing the operation of the metabolic rate measurement system 100 of FIG. 1, in accordance with an illustrative embodiment. FIG. 3 is a diagram of a method 300 of operating the system of FIG. 1 to monitor metabolic rate measurements, in accordance with an illustrative embodiment. FIGS. 2 and 3 are described in conjunction with one another.

As shown in FIG. 2, the timer circuit 120 drives the blood carbon dioxide LED driver circuit 108 to generate a mid-infrared wavelength output via blood carbon-dioxide LED 104 (see FIG. 1). The timer circuit 120 also drives the blood oxygen LED driver circuit 106 to generate a visible and near-infrared wavelength output via LED 102. A blood oxygen photodiode(s) 112 integrated in blood oxygen photodiode circuit 112a receives the output of the LED 106. The output of the blood oxygen photodiode 112 is provided to a sample-and-hold circuit 114 and are digitized by an analog-to-digital conversion circuit to provide a blood oxygen concentration reading (shown as “O₂ concentration value” 202). The blood carbon dioxide photodiode 110 (see FIG. 1) receives the output of the blood carbon-dioxide LED 108. The output of the CO₂ photodiode 110 is amplified via a blood carbon-dioxide preamplifier circuit 118 (e.g., a set of one or more instrumentational amplifier(s)). A lock-in amplifier circuit 116 (e.g., an instrumentational amplifier, balanced modulator, and operational amplifier) amplifies, via the instrumentational amplifier, the input from the blood carbon-dioxide preamplifier circuit 118. The balanced modulator circuit filters noise in the amplified signal based on the frequency of the reference signal, and the operational amplifier amplifies the remaining signal with less noise. The output of the lock-in amplifier is digitized by an analog-to-digital conversion circuit (not shown) to provide a blood carbon-dioxide concentration reading (204).

The system receives (step 302), by the processor 122, on an intermittent basis, at each acquisition, a first set of measurements from one or more non-invasive reflectance- and transmission-based transdermal or transcutaneous sensors (e.g., near-infrared photodiode) located on a wearable, portable, or handheld device placed on or adjacent to a subject in which the first set of measurements is associated with a measure of blood oxygen level. In some embodiments, the one or more non-invasive reflectance- or transmission-based transdermal or transcutaneous sensors comprise one or more first sensors (e.g., a first array of photodiodes) and one or more second sensors (e.g., a second array of photodiodes). The one or more first sensors are configured to interrogate the subject to determine the measure of blood oxygen level. The one or more second sensors are configured to interrogate the subject to determine the measure of blood carbon-dioxide level.

The system 100 receives (step 304), by the processor 122, on an intermittent basis, at each acquisition, a second set of measurements from the one or more non-invasive reflectance- or transmission-based transdermal or transcutaneous sensors (e.g., mid-infrared photodiode) in which the second set of measurements is associated with a measure of blood carbon-dioxide level.

In some embodiments, the first set of measurements associated with the measure of blood oxygen level and the second set of measurements associated with the measure of blood carbon-dioxide level are acquired simultaneously or contemporaneously to one another. In other embodiments, the acquisition of blood carbon dioxide and blood oxygen concentrations can be performed seconds or minutes apart.

As used herein, the term simultaneous acquisition refers to the acquisition that are perform at, or approximate to, a same set of clock cycles used to drive the reflectance- or transmission-based transdermal or transcutaneous sensors.

The system then determines (step 306), by the processor 122, on an intermittent basis, a metabolic rate value 206 based on the received first set of measurements associated with the measure of blood oxygen level and on the received second set of measurements associated with the measure of blood carbon-dioxide level. In some embodiments, the system 100 compares the measured blood carbon-dioxide value and the measured blood oxygen value to a look-up table. In other embodiments, the system 100 computes the metabolic rate value 206 using a transfer function that takes, at least, the measured blood oxygen value and the measured blood carbon-dioxide value as inputs.

The system 100 then outputs (step 308), by the processor 122, at a display 124 of the wearable device, said determined metabolic rate value. In some embodiments, the system stores each of the determined metabolic rate values to allow the stored metabolic rate values to be transmitted to a network (e.g., a cloud service) for subsequent display or analysis. In some embodiments, the system transmit the stored metabolic rate values to a communication device (e.g., smartphone) over a short-range communication channel. The communication device then is configured to transmit the metabolic rate values to a data repository, e.g., in the cloud.

In some embodiments, the system includes long-range communication function and is configured to transmit the data directly to the data repository.

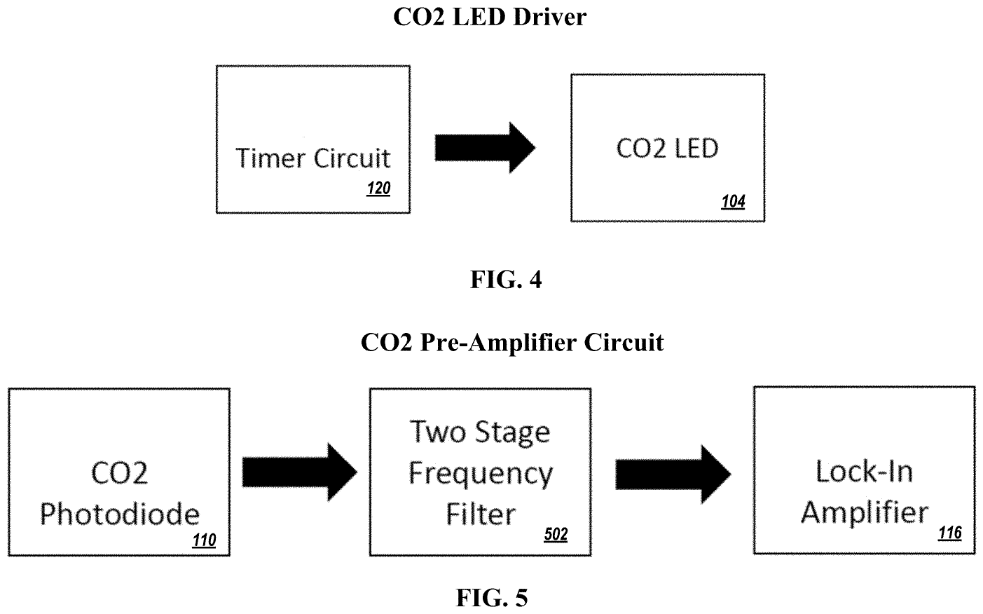

FIG. 4 is a diagram illustrating operation of the blood carbon dioxide LED driver circuit of the system 100 of FIG. 1, in accordance with an illustrative embodiment. The timer circuit 120 generates a timing signal at a pre-defined oscillating frequency and duty cycle to modulate output the blood carbon dioxide LED(s).

FIG. 5 is a diagram illustrating operation of the blood carbon-dioxide pre-amplifier circuit 118 of the system 100 of FIG. 1, in accordance with an illustrative embodiment. The output of the blood carbon dioxide photodiode circuit 110 is outputted to a two-stage frequency filter 502. The first stage is used as a frequency filter to filter self-induced-oscillation noise. The second stage is used to filter self-excitation noise. The output of the pre-amplifier is proportional to blood carbon-dioxide level of the measured subject.

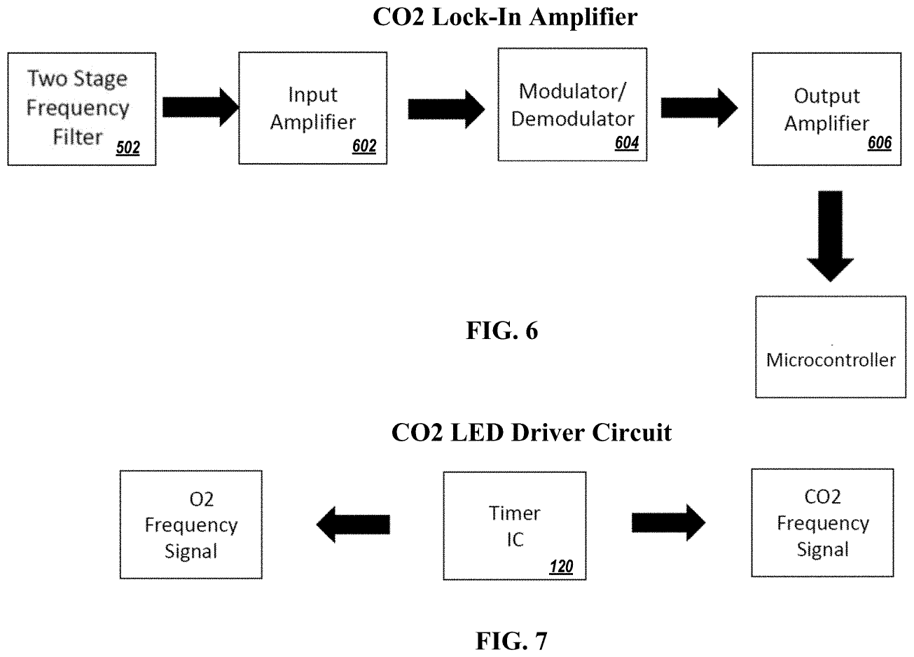

FIG. 6 is a diagram illustrating operation of the blood carbon-dioxide lock-in amplifier circuit 116 of the system 100 of FIG. 1, in accordance with an illustrative embodiment. The blood carbon-dioxide sensor consists of a lock-in amplifier with an LED driver and transimpedance amplifier as the inputs and the blood carbon-dioxide level as the output. The LED driver can be integrated into a microcontroller or other digital circuits serving as the control of the system to drive the blood oxygen sensor with a mid-infrared LED (between about 4.1-4.3 μm peak emission wavelength) that connects to the lock-in amplifier purposed as a reference signal.

As shown in FIG. 6, the blood carbon-dioxide lock-in amplifier circuit 116 includes an input amplifier 602, a modulator and demodulator circuit 604, and an output amplifier 606. The input amplifier 602 receives the output of the preamplifier circuit 118. The input amplifier 602 is configured to amplify the amplitude of the signal. The modulator and demodulator circuit 604 uses the output of the input amplifier 602 and a reference signal to increase the signal-to-noise ratio of the signal. The output amplifier further amplify the output that is provided to an analog-to-digital conversion of a microcontroller.

FIG. 7 is a diagram illustrating an operation of the blood carbon dioxide LED driver circuit 108 of the system of FIG. 1, in accordance with an illustrative embodiment. The blood carbon dioxide LED driver circuit 108 includes the timer circuit 120 that outputs a clock signal to respective LED drive circuits 106, 108 for the blood oxygen and blood carbon dioxide LEDs (102, 104). In some embodiments, the timer circuit is implemented in a microcontroller.

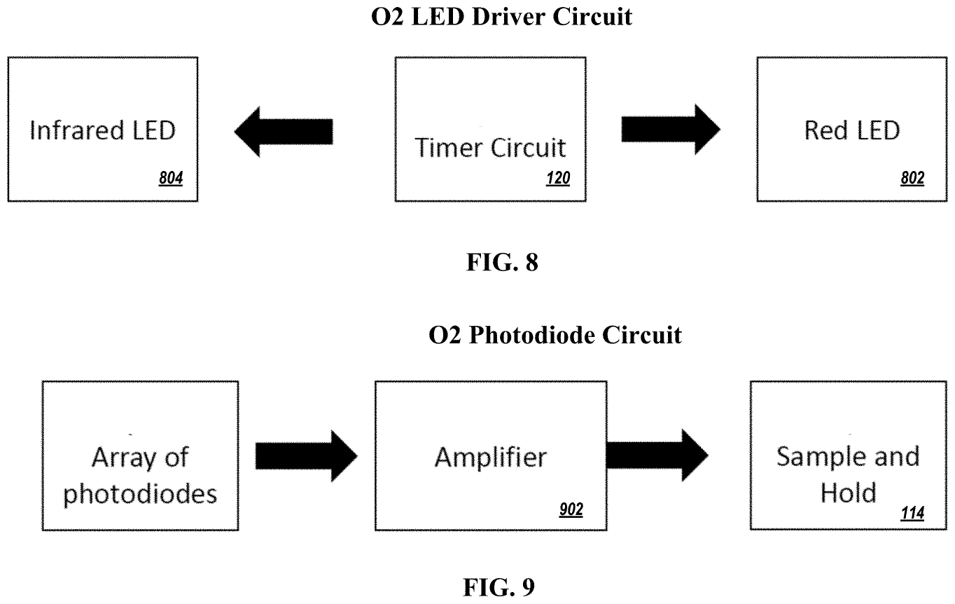

FIG. 8 is a diagram illustrating an operation of the blood oxygen LED driver circuit 106 of the system 100 of FIG. 1, in accordance with an illustrative embodiment. The timer circuit 120 provides an output signal to drive one or more first LED(s) 802 (e.g., configured to output in the red wavelength) and one or more second LED(s) 804 (e.g., configured to output in the near-IR wavelength).

FIG. 9 is a diagram illustrating operation of the blood oxygen photodiode(s) circuit, in accordance with an illustrative embodiment. The current output of the photodiodes (red LED and near-IR LED) are converted, to a voltage signal, and amplified by a transimpedance amplifier 902. The voltage output of the transimpedance amplifier is proportional to the current response of the photodiodes (controlled by the timer circuit).

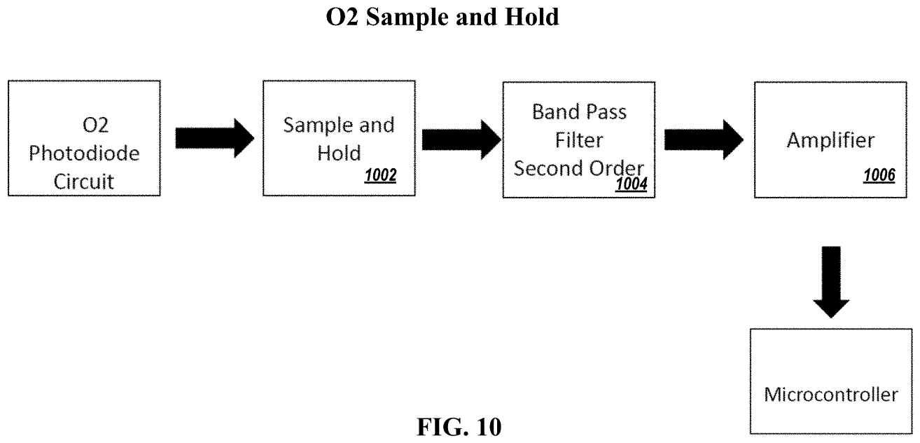

FIG. 10 is a diagram illustrating operation of the blood oxygen sample-and-hold circuit 114 of the system 100 of FIG. 1, in accordance with an illustrative embodiment. The blood carbon dioxide LED driver circuit 108 is connected to the sample-and-hold as a reference signal for the ICs to determine when to sample the signal and when to hold it. The transimpedance amplifier is connected to eight photodiodes connected in parallel to an operational amplifier, which outputs to the sample-and-hold ICs as the receiving input. The sample-and-hold ICs then output a voltage signal that goes through two operational amplifiers configured to filter noise and amplify the remaining signal for subsequent processing.

As shown in FIG. 10, the blood oxygen sample-and-hold circuit 114 includes a sample-and-hold circuit 1002, a band pass filter 1004, and an amplifier 1006. The sample-and-hold circuit 1002 is used to maintain a received voltage outputted from a blood oxygen photodiode circuit for a given sampling period. An example of the sample-and-hold circuit 1002 is the LF398, manufactured by Texas Instruments. The sample-and-hold circuit 1002 reads photodiode output when the LED is off. The band-pass filter 1004 comprises, in some embodiments, a second-order band pass filter that is configured to filter frequencies outside the range of heartbeats. The output of the O₂ sample-and-hold circuit 114 is provided to the microcontroller which determines O₂ concentration from the two acquired channels.

In some embodiments, the system can also detect heart rate from the blood oxygen or blood carbon-dioxide circuits. The system can determine other parameters such as heart variability. The system can use such information (e.g., heart and/or variability) in the determination of metabolic rate and/or co-present the information to provide a more comprehensive view of the body status.

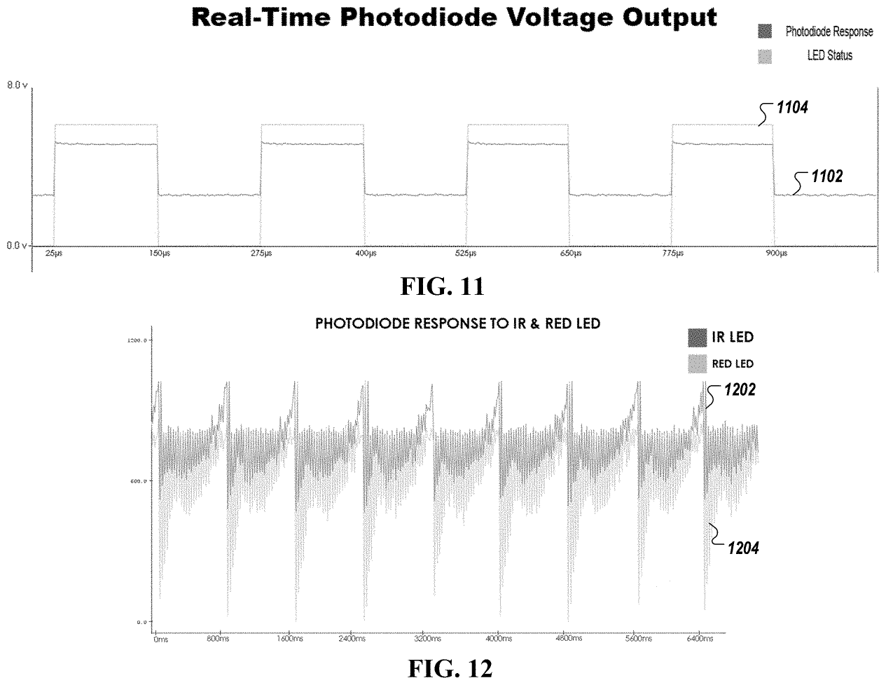

FIG. 11 is a diagram showing experimental results of photodiode responses to pulsed LED of the system 100 of FIG. 1., in accordance to an illustrative embodiment. As shown in FIG. 11, the photodiode response signal 1102 corresponds to the LED output signal 1104. Indeed, each of the outputs of the photodiodes has a fast-response that responds to respective pulsed LED and can be used to track slight blood carbon-dioxide and blood oxygen changes over time.

FIG. 12 is a diagram showing a photodiode sensor response to near- and mid-IR signals in accordance with an illustrative embodiment. A processor can correlate the outputs of the near-IR signal and the mid-IR signal with user's heart rate to help track deviation in blood carbon-dioxide and blood oxygen measurements over time. The photodiode response for the near-IR LED (1202) and red LED (1204) corresponds to one another, though at different amplitudes.

In some embodiments, the current output flows over a sensing load to facilitate sensing as a voltage.

In some embodiments, the LCD is mounted to a wearable device such as a watch having a wrist strap, an attachable pin, a chain (e.g., as a necklace), bracelet, or anklet. In some embodiments, the system 1800 is embedded, or fastened, in an article of clothing (e.g., a shirt, a hat, sunglasses, spectacles etc.).

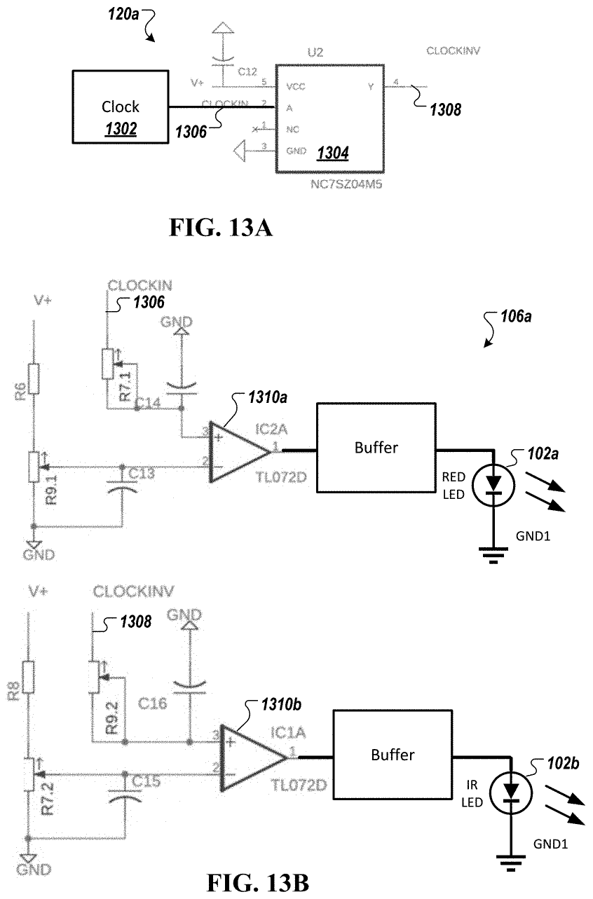

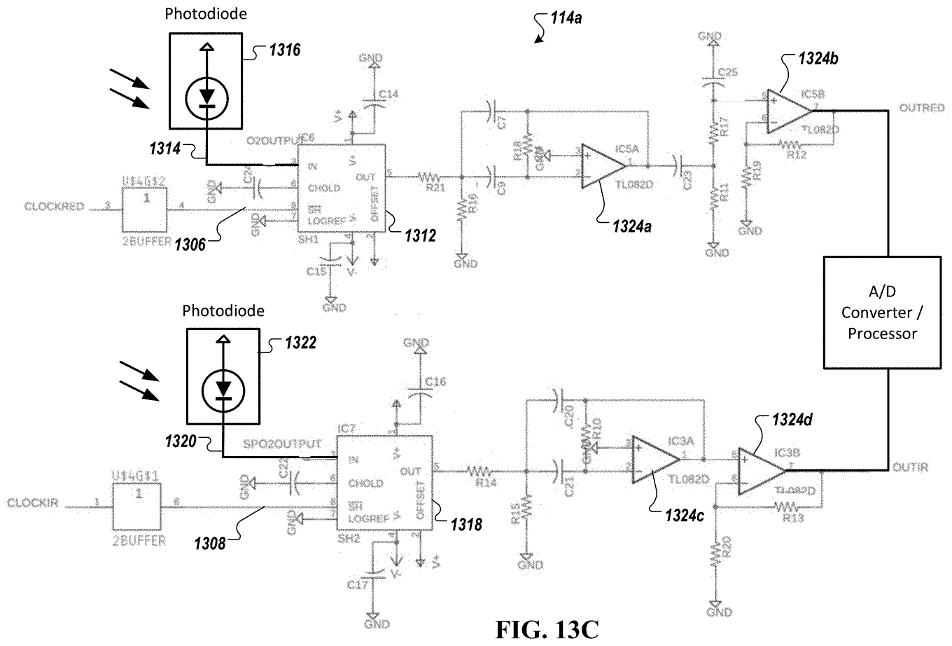

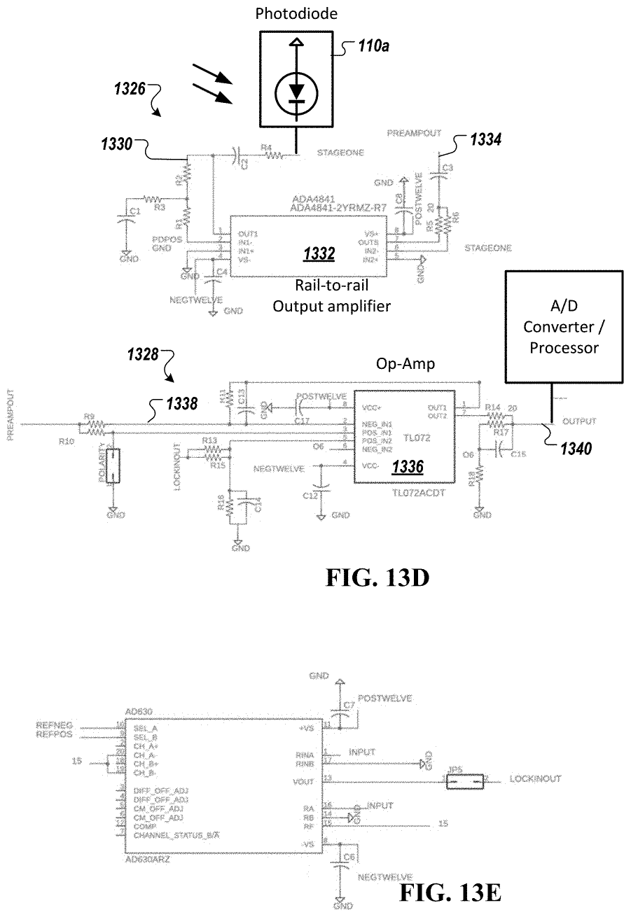

FIGS. 13A, 13B, 13C, 13D, and 13E show example circuit diagram of exemplary system of FIG. 1, in accordance with an illustrative embodiment.

FIG. 13A shows a timer circuit 120 (shown as “120a”) that includes a square-wave signal generator 1302 (shown as a “clock” 1302) and an inverter 1304. The signal generator 1302 provides a clock signal 1306, and the inverter 1304 provides an inverse of that signal (shown as “Clock Inv” 1308). Of course, other methods and circuits of generating complimentary square-wave signals may be used (e.g., using a digital output of a microcontroller and etc., as well as commercially available pulse oximeter circuitries).

FIG. 13B shows an example blood oxygen LED driver circuit 106 (shown as 106a). In FIG. 13B, the driver circuit 106a includes a first amplifier 1310a to drive a first O₂ LED 102 (shown as 102a) comprising a red-LED with the clock signal 1306 and a second amplifier 1310b to drive a second O₂ LED 102 (shown as 102b) comprising an infrared LED with the complementary clock signal 1308. The driver circuit 106a is configured with a variable gain output. In some embodiments, the gain is fixed.

The blood carbon dioxide LED driver circuit 108 and timer circuit 120 may use similar circuitries to drive a mid-range LED 104 associated with blood carbon-dioxide absorption.

FIG. 13C shows an example sample-and-hold circuit 114 (shown as 114a) is configured to provide, e.g., to the microcontroller, a red and infrared signals associated with blood oxygen concentration. The sample-and-hold circuit 114a include a first sample-and-holding circuit 1312 that receives a first signal 1314 from a first photodiode circuit 1316 that includes a photodiode and a current-to-voltage conversion circuit and a second sample-and-holding circuit 1318 that receives a second signal 1320 from a second photodiode circuit 1322 that includes a photodiode and a current-to-voltage conversion circuit. In some embodiments, the same photodiode circuit is used to acquire both the IR and red LED signals.

The first sample-and-holding circuit 1312 is configured to sample its signal input per the sample input, which corresponds to the clock signal 1306, and the second sample-and-holding circuit 1318 is configured to sample its signal input per its sampling input corresponding to the complementary clock signal 1308. The first sample-and-holding circuit 1312 is connected to a set of inverting amplifiers 1324a and 1324b, which provides amplified output of the red-signal to the microcontroller. The second sample-and-holding circuit 1318 is connected to another set of inverting amplifiers 1324c and 1324d, which provides amplified output of the IR-signal to the microcontroller. Voltages of the continuously varying analog signals are held at their respective constant level for a period of time.

Indeed, a current output of the photodiodes (red LED and near-IR LED) are converted, to a voltage signal, and amplified by a transimpedance amplifier (that is proportional to the current response of the photodiodes (controlled by the timer circuit)) and the sample-and-hold circuit outputs a voltage signal that goes through two operational amplifiers configured to filter noise and amplify the remaining signal for subsequent processing. As noted above, multiple red LED and near-IR LEDs may be used, including 1, 2, 3, 4, 5, 6, 7, 8, or more, of each type. The number of red and near-IR LEDs can be the same, or they can be different.

FIGS. 13D and 13E show an example pre-amplifier and lock-in circuits of FIG. 5, in accordance with an illustrative embodiment.

The pre-amplifier circuits of FIGS. 13D includes a first stage amplifier circuit 1326 and a filter circuit 1328. The first stage amplifier circuit 1326 is configured to as a transimpedance amplifier of the input signal from the CO₂ photodiode 110 (shown in CO₂ photodiode circuit 110a). The CO₂ photodiode circuit 110a provides an input signal to the input 1330 of an amplifier 1332 of the stage-one pre-amplifier 1326. In the example, a rail-to-rail output amplifier is shown (part no. ADA4841, manufactured by Analog Device Inc), though other amplifier or instrumentation pre-amplification topology and circuit may be used. The output pin 1334 of that amplifier 1332 is connected to the filter circuit 1328 comprising an operational amplifier 1336. The output pin 1334 of the pre-amplifier op-amp 1332 is connected to the input pin 1338 of operational amplifier 1336 of the filter circuit 1328. The filter circuit 1328 is configured to isolate the main frequency bands of interest. In the instant example, a frequency band of 10 KHzis band-passed by the filter circuit 1328. The output pin 1340 of the band-pass filter is connected to the analog-to-digital conversion circuit and the processor circuit (similar to those of FIG. 13C). Indeed, the first stage amplifier can amplify the amplitude of signal portion of interest (band-passed) and a modulator and demodulator circuit can amplify the signal in reference to a reference signal (i.e., LED source clock signal) to increase the signal-to-noise ratio of the received CO₂ photodiode signal.

The converted signal to digital values of the O₂ signals and the CO₂ signal provides measures of blood carbon-dioxide level and blood oxygen level. The carbon-dioxide concentration values and blood oxygen concentration values are provided to the processor, which can determine metabolic rate value to be presented on a graphical user interface of the system.

Discussion and Examples

In some embodiments, the exemplary device uses LED-based sensors that can detect blood oxygen and blood carbon-dioxide levels through the skin, for instance, in the form of a wrist-worn device. These measures can be transformed via calibration with gold standard indirect calorimetry to provide a measure of metabolic rate. From this measure, a more accurate estimate of daily energy expenditure can be produced compared to estimates based on height, weight, etc.

The blood oxygen sensor is designed to read oxygen levels in the blood stream, similar to a pulse oximeter. The sensor can gather additional information, such as heart rate. The circuit, in some embodiments, consists of sample-and-hold integrated circuits (ICs) and operational amplifiers. The sample-and-hold ICs are designed to retain a voltage, determined by the inputs, to output cleaner signals that are not affected by the rapid change in signals that photodiodes produce. The inputs for the ICs come from an LED driver and a transimpedance amplifier. The LED driver is, in some embodiments, an Arduino designed to drive two LED's, one visible LED and one near-infrared LED with wavelengths 660 and 940 nanometers, respectively. The driver is connected to the sample-and-hold as a reference signal for the ICs to determine when to sample the signal and when to hold it. The transimpedance amplifier consists of eight photodiodes connected in parallel to an operational amplifier, which outputs to the sample-and-hold ICs as the receiving input. The sample-and-hold ICs then output a voltage signal that goes through two operational amplifiers designed to filter some noise and amplify the remaining signal for future processing.

The blood carbon-dioxide sensor consists, in some embodiments, of a lock-in amplifier with an LED driver and transimpedance amplifier as the inputs and the blood carbon-dioxide concentration as the output. The LED driver can be the same microcontroller (e.g., Arduino) that drives the blood oxygen sensor, with a mid-infrared LED (e.g., at around 4.1-4.3 μm peak emission wavelength) that connects to the lock-in amplifier purposed as a reference signal. The lock-in amplifier consists, in some embodiments, of an instrumentational amplifier, balanced modulator, and operational amplifier. The instrumentational amplifier amplifies the input from the transimpedance amplifier, the balanced modulator filters the noise based on the frequency of the reference signal, and the operational amplifier amplifies the remaining signal with less noise. The output of the lock-in amplifier is then used for analysis.

After the blood oxygen and carbon-dioxide levels are determined, the respiratory quotient is found. The data collected from the sensor and respiratory quotient are used and calibrated with results from an indirect calorimetry test to output a metabolic rate. Additional data can be gathered through user input to compare statistical values from other users versus the user's values.

Aliasgharzdeh et al., “Comparison of Indirect calorimetry and Predictive Equations in Estimating Resting Metabolic Rate in Underweight Females,” Iran J Public Health. 2015 June; 44(6): 822-829 describes the use of predictive equations in estimating resting metabolic rate based on measured oxygen consumption. Rather than resting metabolic rate, the exemplary system can determine body metabolic rate based on both oxygen and carbon dioxide concentration.

The data gathered from the sensor system can have multiple applications. In the medical industry, practitioners can use the exemplary device to assess and track patient nutrition. This could be useful for metabolic disorders (i.e., diabetes), weight management, and general nutrition analysis. In some embodiments, the system can output heart rate, heart rate variability, blood oxygen levels, blood carbon-dioxide levels, metabolic rate, and energy expenditure. Practitioners could use the exemplary device to continuously monitor these parameters at the bedside.

In the sports industry, professional athletes can use the information to monitor diets and health for the purpose of adjusting diets and exercise routines. Currently, athletes may need to use inconvenient and expensive methods such as indirect calorimetry to track their metabolism and adjust their diets accordingly. With the device's ability to track metabolic rates in real-time, athletes will know when their metabolic rates are at their highest and lowest, allowing them to reach their peak performance naturally.

On the consumer level, individuals can use the exemplary device for weight management and fitness applications. The exemplary device will provide the user with insights into their diet and fitness routine.

In some embodiments, the system is configured to continuously monitor the metabolic rate of a patient throughout the day. In such system, the system may acquire a series of measurement, say, about every 15 minutes, or some pre-defined intervals (e.g., every 10 minutes, ½ hours, ¾ hours, 1 hours). In each measurement, multiple acquisition of O₂ and CO₂ may be acquired and the mode and/or mean of the multiple acquisitions may be determined (e.g., for subsequently presentation or analysis).

In some embodiments, the system is configured to monitor the metabolic rate of a patient based on a trigger (e.g., when triggered by a certain level of movement of activity, say, from accelerometer measurements).

In some embodiments, the system is configured to vary measurements based on a determination the patient is as sleep. In some embodiments, the determination of sleep may be based on other sensor inputs. In some embodiments, the system may use the measured metabolic rate to determine sleep activity and maybe adjust frequency of measurements accordingly (e.g., reducing the frequency of measurements as a means to improve battery and energy usage management).

In some embodiments, the system is configured to monitor the metabolic rate when prompted by the user. In such embodiment, the system may initiate a new measurements and present such measurement of the user. In other embodiments, the system may present to the user the last measured metabolic rate measurement. In some embodiments, the system may present to the user the last measured metabolic rate measurement and indicate that a new measurement is being acquired.

Indeed, whether for health issues, dietary reasons, or fitness regimens, awareness of caloric intake and expenditure is sought out by a variety of individuals. The exemplary system and methods of determining metabolic rate improves over other methods of using environmental chambers (direct calorimeters), indirect calorimeters, and/or estimations based on population-level data. Environmental chambers and indirect calorimeters provide high accuracy measurements and are generally only be accessible in clinical or athletic training settings. Although estimations based on population-level data are easily accessible, they also tend to be inaccurate to a certain degree. To increase access to high accuracy, metabolic measurements, the exemplary method and system provides a portable, optical transdermal or transcutaneous sensor system to monitor blood gas concentrations. The exemplary system and method can be used in combination with environmental chambers (direct calorimeters), indirect calorimeters, and/or estimations based on population-level data to provide another set of information.

With the instant exemplary system and method, metabolic rate are determined using similar methods to indirect calorimetry, but without complexity of such breathing associated instrumentation. Healthy individuals can utilize this metabolic insight to optimize diet and activity; while, individuals with metabolic disorders (i.e. diabetes, obesity) can partner with their medical providers to best use these metabolic insights in condition management and weight loss.

Referring back to FIG. 1, in some embodiments, the exemplary system 100, in some embodiments, includes a wireless transceiver 128. As shown in FIG. 1, in this example, the wireless transceiver 128 is coupled to the electronic circuitry (e.g., processor 1810) and is configured to transmit, over a communication channel, the generated metabolic rate parameter to another computing device (not shown). In some embodiments, the transmission is over a near-field communication channel (e.g., Bluetooth, Wi-Fi, infrared, and the like) to facilitate presentation of the metabolic data to a person, e.g., on a portable computing device and portable computing watch) or over a combination of near-field and far-field communication channel to store, e.g., in the cloud. A cloud-based application may send notification and alerts based on the measured metabolic rate value and corresponding data generated by the processor.

While the methods and systems have been described in connection with preferred embodiments and specific examples, it is not intended that the scope be limited to the particular embodiments set forth, as the embodiments herein are intended in all respects to be illustrative rather than restrictive.

As used herein, “computing device” may include a plurality of computers. The computers may include one or more hardware components such as, for example, a processor, a random access memory (RAM) module, a read-only memory (ROM) module, a storage, a database, one or more input/output (I/O) devices, and an interface. Alternatively, and/or additionally, controller may include one or more software components such as, for example, a computer-readable medium including computer executable instructions for performing a method associated with the exemplary embodiments. It is contemplated that one or more of the hardware components listed above may be implemented using software. For example, storage may include a software partition associated with one or more other hardware components. It is understood that the components listed above are exemplary only and not intended to be limiting.

Processor may include one or more processors, each configured to execute instructions and process data to perform one or more functions associated with a computer for indexing images. Processor may be communicatively coupled to RAM, ROM, storage, database, I/O devices, and interface. Processor may be configured to execute sequences of computer program instructions to perform various processes. The computer program instructions may be loaded into RAM for execution by processor. As used herein, processor refers to a physical hardware device that executes encoded instructions for performing functions on inputs and creating outputs.

A processor can be microcontrollers, microprocessors, or logic circuits such as ASICs (Application Specific Integrated Circuit), CPLDs (Complex Programmable Logic Device), FPGA (Field Programmable Gate Array), or other programmable logic integrated circuits. In some embodiments, a processor is configured to execute instruction stored in a memory of the device.

RAM and ROM may each include one or more devices for storing information associated with operation of processor. For example, ROM may include a memory device configured to access and store information associated with controller, including information for identifying, initializing, and monitoring the operation of one or more components and subsystems. RAM may include a memory device for storing data associated with one or more operations of processor. For example, ROM may load instructions into RAM for execution by processor.

Storage may include any type of mass storage device configured to store information that processor may need to perform processes consistent with the disclosed embodiments. For example, storage may include one or more magnetic and/or optical disk devices, such as hard drives, CD-ROMs, DVD-ROMs, or any other type of mass media device.

Database may include one or more software and/or hardware components that cooperate to store, organize, sort, filter, and/or arrange data used by controller and/or processor 122. For example, database may store hardware and/or software configuration data associated with input-output hardware devices and controllers, as described herein. It is contemplated that database may store additional and/or different information than that listed above.

I/O devices may include one or more components configured to communicate information with a user associated with controller. For example, I/O devices may include a console with an integrated keyboard and mouse to allow a user to maintain a database of images, update associations, and access digital content. I/O devices may also include a display including a graphical user interface (GUI) for outputting information on a monitor. I/O devices may also include peripheral devices such as, for example, a printer for printing information associated with controller, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface may include one or more components configured to transmit and receive data via a communication network, such as the Internet, a local area network, a workstation peer-to-peer network, a direct link network, a wireless network, or any other suitable communication platform. For example, interface may include one or more modulators, demodulators, multiplexers, demultiplexers, network communication devices, wireless devices, antennas, modems, and any other type of device configured to enable data communication via a communication network.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of monitoring metabolic rate measurements, the method comprising:
receiving, by a processor, on an intermittent basis, at each acquisition, a first set of one or more measurement values derived from measurements using a first non-invasive reflectance- or transmission-based transdermal or transcutaneous sensor located on a device placed on or adjacent to a subject, wherein the first set of one or more measurement values is associated with a measure of blood oxygen concentration, and wherein the first non-invasive reflectance- or transmission based transdermal or transcutaneous sensor comprises visible and near-infrared photodiodes;
determining, by the processor, blood oxygen concentration value from one or more of the first set of measurements;
receiving, by the processor, on an intermittent basis, at each acquisition, a second set of measurements from the one or more non-invasive reflectance- or transmission-based transdermal or transcutaneous sensors located on the device placed on the subject, wherein the second set of one or more measurement values is associated with a measure of blood carbon-dioxide concentration, and wherein the second non-invasive reflectance- or transmission-based transdermal or transcutaneous sensors comprises a mid-infrared photodiode;
determining, by the processor, a blood carbon-dioxide concentration value from one or more of the second set of measurements;
determining, by the processor, on an intermittent basis, a metabolic rate value based on the determined blood oxygen concentration value and the determined blood carbon-dioxide concentration value; and
outputting, by the processor, at a display of the device, said determined metabolic rate value.

2. The method of claim 1, further comprising:
storing, by the processor, each of the determined metabolic rate values; and
transmitting, by the processor, the stored metabolic rate values to a network for subsequent display or analysis.

3. The method of claim 1, wherein the second non-invasive reflectance- or transmission-based transdermal or transcutaneous sensors are part of an array of photodiodes.

4. The method of claim 1, wherein the first non-invasive reflectance- or transmission-based transdermal or transcutaneous sensors are part of an array of photodiodes.

5. The method of claim 1, wherein the first set of one or more measurement values associated with the measure of blood oxygen concentration and the second set of one or more measurement values associated with the measure of blood carbon-dioxide concentration are acquired simultaneously to one another.

6. The method of claim 1, wherein the visible and near-infrared LEDs are configured to output predominantly at around a 660-nm wavelength and a 940-nm wavelength, respectively.

7. The method of claim 1, wherein the mid-infrared LED is configured to output predominantly at around a 4.2-μm wavelength.

8. The method claim 1, wherein the outputted metabolic rate values are used to assess and track subject's nutrition, metabolic disorders, activity level, or weight management.

9. The method of claim 1, further comprising:
determining, by the processor, on an intermittent basis, at each acquisition, a heart rate value from the received first set of measurements associated with the measure of blood oxygen concentration or from the received second set of measurements associated with the measure of blood carbon dioxide concentration; and
outputting, by the processor, at the display of the wearable, portable, or handheld device, said determined heart rate value (e.g., concurrently or separately with the outputted metabolic rate value).

10. The method of claim 9, further comprising;
determining, by the processor, on an intermittent basis, a parameter selected from the group consisting of a heart rate variability parameter and an energy expenditure parameter; and
outputting, by the processor, at the display of the device, said parameter.

11. A system comprising:
one or more reflectance- or transmission-based transdermal or transcutaneous sensors, including a first reflectance- or transmission-based transdermal or transcutaneous sensor comprising visible and near-infrared photodiodes having wavelengths associated with blood oxygen concentration and a second reflectance- or transmission-based transdermal or transcutaneous sensor comprising a mid-infrared photodiode associated with blood carbon-dioxide concentration;
a processor operatively connected to the one or more reflectance- or transmission-based transdermal or transcutaneous sensor s and the display; and
a memory operatively connected to the processor, the memory having instructions stored thereon, wherein execution of the instructions by the processor, cause the processor to:
receive a first set of one or more measurement values derived from measurement of the first non-invasive reflectance- or transmission-based transdermal or transcutaneous sensor having been placed on a first surface location of a person;
determine blood oxygen concentration value from the first set of measurements;
receive a second set of one or more measurement values from measurement of the second non-invasive reflectance- or transmission-based transdermal or transcutaneous sensor;
determine a blood carbon-dioxide concentration value from the second set of measurements;
determine a metabolic rate value based on the determined blood oxygen concentration value and the determined blood carbon-dioxide concentration value; and
output at the display said determined metabolic rate value.

12. The system of claim 11, wherein the instructions, when executed by the processor, further cause the processor to:
store each of the determined metabolic rate values; and
transmit the stored metabolic rate values to a network for subsequent display or analysis.

13. The system of claim 11, wherein the second non-invasive reflectance- or transmission-based transdermal or transcutaneous sensors are part of an array of photodiodes.

14. The system of claim 11, wherein the first non-invasive reflectance- or transmission-based transdermal or transcutaneous sensors are part of an array of photodiodes.

15. The system of claim 11, wherein the first set of one or more measurement values associated with the measure of blood oxygen concentration and the second set of one or more measurement values associated with the measure of blood carbon-dioxide concentration are acquired simultaneously to one another.

16. The system of claim 11, wherein the visible and near-infrared photodiodes are configured to output predominantly at around a 660-nm wavelength and a 940-nm wavelength, respectively.

17. The system of claim 11, wherein the mid-infrared photodiode is configured to output predominantly at around a 4.2-μm wavelength.

18. The system of claim 11, wherein the instructions, when executed by the processor, further cause the processor to:
determine a heart rate value from the received first set of measurements associated with the measure of blood oxygen concentration or from the received second set of measurements associated with the measure of blood carbon dioxide concentration; and
output at the display said determined heart rate value.

19. The system of claim 18, wherein the instructions, when executed by the processor, further cause the processor to:
determine a parameter selected from the group consisting of a heart rate variability parameter and an energy expenditure parameter; and
output at the display of the device said parameter.

20. A non-transitory computer readable medium having instructions stored thereon, wherein execution of the instructions, when executed by the processor, cause the processor to:
receive a first set of one or more measurement values derived from measurement of the first non-invasive reflectance- or transmission-based transdermal or transcutaneous sensor having been placed on a first surface location of a person, wherein the first reflectance- or transmission-based transdermal or transcutaneous sensor comprises visible and near-infrared photodiodes having a wavelength associated with blood oxygen levels;
determine blood oxygen concentration value from the first set of measurements;
receive a second set of one or more measurement values from measurement of the second non-invasive reflectance- or transmission-based transdermal or transcutaneous sensor, wherein the second reflectance- or transmission-based transdermal or transcutaneous sensor comprises a mid-infrared photodiode associated with blood carbon-dioxide concentration levels;
determine a blood carbon-dioxide concentration value from the second set of measurements;
determine a metabolic rate value based on the determined blood oxygen concentration value and the determined blood carbon-dioxide concentration value; and
output at the display said determined metabolic rate value.
